(19) United States
(12) Patent Application Publication
(10) Pub. No.: US 2011/0288379 A1
(43) Pub. Date: Nov. 24, 2011

(54) BODY SIGN DYNAMICALLY MONITORING SYSTEM

(75) Inventor: Jiankang Wu, Beijing (CN)

(73) Assignee: WUXI MICROSENS CO., LTD., Wuxi (CN)

(21) Appl. No.: 12/671,523

(22) PCT Filed: Jul. 17, 2008
(86) PCT No.: PCT/CN08/01332

§ 371 Date: Jul. 26, 2011

Related U.S. Application Data
(63) Continuation of application No. PCT/CN2008/001332, filed on Jul. 17, 2008.

(30) Foreign Application Priority Data
Aug. 2, 2007 (CN) 200710119869.8

(51) Int. Cl. A61B 5/00

(52) U.S. Cl. 600/301

(57) ABSTRACT

A real-time body status monitoring system (RBMS) is presented in this invention. A wearable monitoring apparatus (WMA) worn by users consists of one or a few sensor nodes and a computing module. Sensor nodes communicate with the computing module via either wired or wireless protocols. RBMS incorporates a monitoring center that connects and serves many WMAs. Together with the sensors and context-aware information fusion and analysis, the system in the invention goes beyond sampling rare events that may be of profound diagnostic, prognostic, or therapeutic importance. It measures the physiological responses to therapeutic interventions during daily activities, which constitute direct and practical health indicators for the patient.

### BACKGROUND OF THE INVENTION

1. Field of the Invention

The present invention relates to medical and healthcare instruments and specifically relates to real-time body status monitoring in daily life, daily care techniques, and context-aware analysis for body status estimation.

2. Description of the Related Art

According to the 2005 Beijing Cardiovascular Disease Forum, the number of cardiovascular disease patients in China has increased four-fold to become China's number one killer. The economic loss from cardiovascular disease in China is estimated at 300 billion yuan. In the US, about one-quarter of Americans 70 million people—suffer from cardiovascular disease, which causes direct and indirect losses of US$393 billion.

Experts (Philip F. Binkley, “Predicting the Potential of Wearable Technology,” IEEE Engineering in Medicine and Biology, May/June 2003, pp. 23-24) believe that wearable technologies will create a revolution in the management of cardiovascular disease. Such technologies permit diagnosis and monitoring at home or at work, reduce risk, restore labor productivity, and meet elderly care needs. As a result, hospitalization and mortality rates would be greatly reduced. Within China and the US alone, there are around 200 million potential consumers for such technologies.

Existing wearable medical devices are not able to perform dynamic monitoring and diagnosis. China Patent No. 200510036412.1 describes an internet-based, personal electrocardiogram (ECG) system consisting of an ECG detecting module, processing module, and data transfer module. It permits real-time ECG monitoring by a remote professional. A series of US patents (such as U.S. Pat. No. 6,665,385 and U.S. Pat. No. 6,225,901 by Cardio Net Company and U.S. Pat. No. 6,611,705 by Motorola) provide similar services. However, these devices do not measure the wearer's situational information, which is crucial to interpreting the ECG data. For example, a heart rate of 120 is abnormal when sitting at rest, but normal when exercising. Our proposed device collects data from three types of sensors: physical activity, environmental stimuli, and ECG.

U.S. Pat. No. 5,606,978 discloses an ambulatory heart monitoring apparatus with an IC card. The apparatus records EGG signals from the electrodes onto the IC card together with operational data (such as battery voltage) and calibration data, which are processed at a remote processing station. Similarly, U.S. Pat. Nos. 4,519,398 and 4,211,238 record heart rate, blood pressure, and time. The analysis and printing of data are performed in clinics.

The limitations of existing medical instruments in hospitals or clinics are that they measure patients in a lab over a short period of time, and the patients are asked to sit still. These instruments capture neither rare events, nor the dynamic status of people in their daily life: at work or on the move. Although wireless ECG and Holter technologies are designed for continuous monitoring, they do not capture contextual information, and, therefore, are not able to perform real-time assessment and diagnosis. It is impossible to perform real-time diagnosis during daily activities without contextual information. Real-time capture and analysis of physiological signals together with contextual information give an accurate status report and performance assessment of a person's physical well-being. This type of device will play increasingly more important roles in healthcare services.

### BRIEF SUMMARY OF THE INVENTION

To overcome the uncertainties that currently obscure the relationship between a person's physiological status and situational information (intensity of activity, environment, and physiological status), this invention aims to continuously monitor physiological signs, collecting the bodily signals associated with both rare and quotidian events alongside related situational conditions. Thus, the invention provides a wearable real-time body status monitoring system (RBMS) that captures and analyzes a person's dynamic physiological responses and rhythms.

To achieve the aforementioned objectives, the architecture of the RBMS is as follows:

A RBMS consists of one monitoring center and many wearable monitoring apparatuses (WMAs), each of which is worn by a user. The WMAs comprise one or more sensor nodes and a computing module. The sensor nodes of the WMA fall into two categories of sensors: sensors in one category monitor physiological signals, and sensors in another category monitor situational signals that may influence and explain a person's physiological status.

The computing module gathers data from the sensor node(s) either via wired or wireless connections and receives, processes, stores, and analyzes the signals, including physiological signals such as ECG, temperature, activity signals from an accelerometer or gyroscope, environmental signals, such as temperature and noise levels, and physiological signals, such as skin conductivity levels. The computing module also manages and controls the sensor nodes and provides a human-machine interaction interface.

A monitoring center communicates with the computing module of the WMAs via either a wired or wireless connection. The center receives, processes, stores, and fuses data from computing modules worn by different users and provides information, data analysis, and mining tools to doctors and caregivers for their research and consultation services. Via the monitoring center, doctors may perform extended patient studies and send out medication instructions.

The embodiments of the invention include the aforementioned two categories of sensors consisting of:
(1) Physiological sensors may include, but are not limited to, heart rate meters, ECG, blood pressure meters, oxyhemoglobinometers, thermometers, respirometers, electroencephalographs, and glucometers.
(2) Situational sensors fall into three subcategories:
   1) Sensors that capture body motion and activities. These sensors may include, but are not limited to, accelerometers, gyroscopes, tensiometers that detect joint motion, and video cameras.
   2) Environment sensors, including, but not limited to, temperature, noise, atmospheric pressure, and location (i.e., GPS).
   3) Sensors that measure physiological status, such as skin conductivity, electroencephalogram (EEG) sensors, and microphone recording of events that may affect emotions.

The embodiments of the invention include the aforementioned computing module, which consists of:
(1) A set of preamplifiers and analog-to-digital converters (ADC) that receive signals collected by all sensors, amply those signals to meet the ADC requirements, then convert those analog signals to digital form.
(2) A set of sensor signal fusion and analysis units (SFAs) that receive digital signals from the ADC, process and fuse the parallel sensor signals, and store all signal data in a local (on-chip) database. The result is a context-aware multi-sensor information fusion unit (CIF) that gathers and records the many time-dependent data streams from the wearer.
(3) A CIF receives inputs from the SFA, fuses the sensor information from multiple sensors, performs a context-aware analysis, and estimates the body's status, providing, as output, possible indications of abnormal physiological events together with a temporal history consisting of aligned multiple sensory data streams that show the temporal development of the abnormal signal and the contextual information that may (or may not) have contributed to the abnormal physiological event.
(4) A human-machine interaction unit (HMI) displays the results from the SFA, CIF, local monitoring database (LMDB, described below), and local systematic database unit (LSDB), receives and responds the user requests, and displays information from the monitoring center to caregivers or family members.
(5) A LMDB stores short-term (i.e., days, weeks, or months) data: the original sensory data, processing and analysis results from the SFA and CIF, a personal profile and health history, and service definitions and parameters, such as warning and reminder thresholds.
(6) A LSDB stores the sensor node and computing module system parameters and their runtime status.

The embodiments of the invention include the aforementioned monitoring center, which consists of:
(1) A context-aware diagnosis and service unit (CDS) that receives, stores, fuses information from various WMAs, and performs analysis and data mining across time, users, age group, sex, and data modalities, providing a platform through which medical experts may conduct research, diagnose disease, and monitor patients as a component of advisory services.
(2) A central database (CDB) stores long-term copies of data from all WMAs and for all users throughout the period of service usage. The CDB stores the original sensor data, analysis results, personal profiles, health history, diagnoses, therapy plans, therapy results, service definitions, and parameters for all WMAs.
(3) A central system management database (CSDB) stores all system parameters, the runtime status of all WMAs, and the status of the monitoring center system.

Included in the embodiments of the invention is a functionality such that if the system or service parameter inputs of the LMDB, which monitors the WMAs, reach predefined threshold values, alerts or reminders will be triggered in accordance with predefined protocols in the database.

According to the embodiments of the invention, the aforementioned system computing module modifies the system parameters when receiving a command from the CDB of the monitoring center during database synchronization.

According to the embodiments of the invention, the aforementioned system LMDB in the computing module synchronizes its data with the CDB in the monitoring center, while the CDB synchronizes the newly defined system and service parameters with the relevant LMDB. All synchronizations are event-driven.

According to the embodiments of the invention, the aforementioned WMAs consist of a computing module and one or more sensor modules, connected either by wire or wirelessly to achieve seamless communication. The computing module communicates with the monitoring center. In the embodiment of this invention:
(1) Sensor nodes consist of one or more sensors, preamplifiers, ADCs, wired/wireless communication modules, microcontrollers, and power management modules, which are integrated into an embedded or on-chip system.
(2) The computing module is implemented by integrating the SFAs, the CIFs, the HMIs, the LMDBs, and the LSDB as an integrated system that runs on a personal digital assistant (PDA) or smart phone-type of device.
(3) If the sensor node is sufficiently powerful, the SFA may be moved from the computing module to the sensor node. Sophisticated preprocessing of the sensor signals would be implemented in such a case. In this case, the amount of data transmitted from the sensor node to the computing module would be greatly reduced because only a subset of raw data samples would need to be associated with and recorded alongside the processed results.

According to the embodiments of the invention, another implementation scheme includes:

A computing module that is implemented on a dedicated wearable microcomputer such that all sensors are directly connected to the microcomputer. The microcomputer is connected to the monitoring center via wired or wireless communication.

According to the embodiments of the invention, another implementation scheme is: The sensor node includes powerful computing capabilities that include the SFA and CIF capabilities, while a commercially available PDA or smart phone is used as a platform for the user interface, local database, and communication module connecting both the sensor node and the central monitoring center.

According to the embodiments of the invention, the aforementioned SFAs acquire, process, analyze, and fuse multiple sensor signals of the same type to generate a meaningful interpretation of the data stream. For example, fusion of multiple accelerometer signals from different segments of the body may be used to deduce an activity type and step frequency; analysis of ECG signals from various electrodes on different parts of the body permit derivation of the heart rate and detection of abnormal beats.

According to the embodiments of the invention, the aforementioned CIF performs context-aware sensor data fusion to estimate a body's health status by analyzing physiological sensor signals in relation to the contextual and situational sensory data. Here, the contextual and situational information consists of physical and mental activity, environmental conditions, and physiological status.

According to the embodiments of the invention, the aforementioned CDSs are implemented in the monitoring center. CDSs continuously collect and analyze physiological signals, the corresponding contextual signals, and the fusion and analysis results from each user's WMA. Long-term, complete, and continuous data and information collection and analysis for a variety of users will enable development of novel diagnostic methods. Over time, this system will build a long-term continuous monitoring profile for each user, identify physiological circadian rhythms and changes in individuals, and discover the factors that influence a person's physiological status along the dimensions of individual, age, sex, activity, environment, physiology, and other contextual conditions.

According to the embodiments of the invention, when a monitoring center is unavailable, the user can access his own body status at any time through his WMA. The WMA sends reminders and alerts, checks the system status, and alerts the user if an electrode is reporting poor contact performance or if the battery requires recharging. The WMA stores data and analysis results for days, weeks, and months.

According to the embodiments of the invention, the WMA becomes a continuous ECG monitoring device when only ECG sensors are present and operating; the WMA becomes an activity monitoring device that classifies activity types, measures the intensity of activity, estimates the energy consumption, and advises the user of the appropriate quantity of exercise if only accelerometers or gyroscopes are present and operating; the WMA becomes a real-time localization device if only GPS or other localizer capabilities are present and operating; the WMA becomes a lie-detector or mood measuring device if only skin conducting sensors are present and operating.

According to the embodiments of the invention, the aforementioned WMA has the following human-machine interacting and self-maintenance functions: time-keeping functions, automatic signal acquisition, signal processing, personalized analysis and alerting systems, communication and networking capabilities, self-maintenance, performance optimization routines, e.g., the system is able to reorganize its functions and tune system parameters if the architecture or resource loads change, such as sensor addition or removal, or a drop in the battery charge level.

According to the embodiments of the invention, a simplified version of the aforementioned WMA constitutes a health care device for exercise and lifestyle monitoring rather than a medical instrument. This simplified device consists of an ECG sensor, accelerometers, respiration sensor, an environmental thermograph, or a subset of these sensors. This device contains functions that measure a cardiovascular fitness index, monitor exercise intensity, evaluate the physiological effects of exercise, detect any possible abnormal ECG signals, analyze heart dynamics (heart rate and ECG waveform variations against activity intensity), and remind the user if arrhythmias or other abnormalities occur during exercise.

According to the embodiments of the invention, the aforementioned users of WMAs can have access to an online community. In the community, users can create accounts, acquire storage space, retrieve their own analyzed data, and share the data with friends using tools provided by the community server. Additionally, users are able to choose and contact professional consultants online, leave messages, find friends, and organize discussions and online seminars. This online community serves as a platform for professional consultations and interest group activities.

According to the embodiments of the invention, the aforementioned WMAs communicate with the online community via wireless communication to upload user data to the online storage space and to automatically upgrade software. Users can log on to their accounts for account management and/or profile updating.

This invention, namely, RBMS, has the following unique features that existing hospital and clinic medical instruments do not have:
(1) Capable of sampling rare events that may be of profound diagnostic, prognostic, or therapeutic importance;
(2) Able to measure physiological responses during normal periods of activity, rest, and sleep, and in certain environmental conditions. These responses are practical health indicators for the patient and enable a patient to respond to changing conditions or seek therapeutic intervention;
(3) Able to capture an individual's circadian signals and variations in these physiologic signals that correlate with the progression of disease.

The invention, RBMS, encompasses a set of functions, including wearing, connecting, and managing sensors, collecting and processing sensor data, classifying and describing physical activities, processing environmental and physiological signals, fusing the physiological and contextual information (activity, environment, and physiological status) to estimate the bodily health status, predicting a condition's onset, providing precautions, warnings, and reminders to the user, connecting and synchronizing the WMA with the monitoring center, implementing data storage and data management systems, and coordinating medical services in response to physiological monitoring activities. Through continuous collection and analysis of physiological signals, human activity levels, and environmental conditions, the system dynamically monitors and permits diagnosis and therapy throughout daily life, instead of relying on static measurements in hospitals and clinics. As a component of a new generation of medical instruments, this invention gathers data and provides a means for growing novel medical diagnosis and therapy methods that can reduce hospitalization and mortality rates.

The person-to-person variations and variability of heart rate within an individual, blood pressure fluctuations, and other physiological parameters as a function of the time of day, day of the month, or over longer time scales constitutes significant indicators of trends in bodily health and the progression of certain diseases. Tracking variations may permit identification of the best time of day or day of the month at which medications should be administered. Novel effective diagnostic and treatment methods can result from widespread use of this invention, namely, by using WMAs and the RBMS. In contrast with “static measurement and diagnosis” methods implemented in hospitals and clinics, we refer to this new method as “dynamic measurement and diagnosis.” This invention can be applied to health monitoring services by providing a feedback-driven basis for advising services that suggest more effective exercise regimens or lifestyle changes, and help measure a person's individual response to their environment.

### BRIEF DESCRIPTION OF THE DRAWINGS

An embodiment of the present invention will now be described, by way of example, with reference to the accompanying drawings, in which:

FIG. 1 shows the architecture and the overall block diagram of this invention, namely, RBMS.

FIG. 2 shows the embodiment of this invention, namely, RBMS.

FIG. 3 shows the processes and detailed block diagram of RBMS.

FIG. 4 illustrates the context-aware multi-sensor information fusion for real-time assessment of the heart status using Bayesian network dynamic systems theory.

FIG. 5 shows an implementation scheme for the WMA described by this invention.

FIG. 6 shows another implementation scheme for the WMAs described by this invention.

### DETAILED DESCRIPTION OF THE INVENTION

A detailed description of this invention with figures will be given as follows. The embodiment is provided here with the goal of facilitating an understanding of this invention rather than imposing any limitations on the scope of the invention.

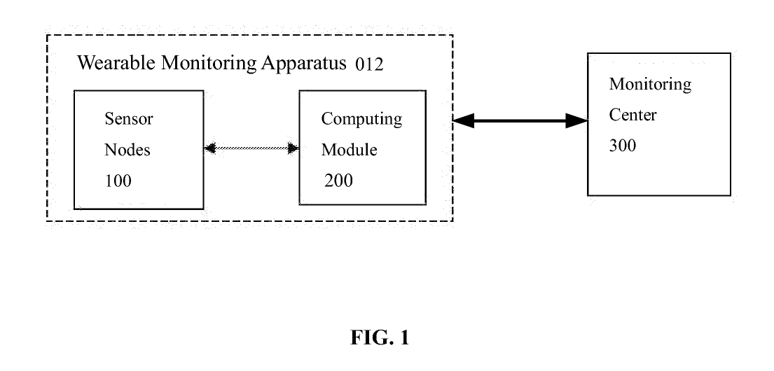

FIG. 1 shows an overall block diagram that includes the structure of this invention, namely, the RBMS. This invention consists of hardware and software components that form a type of body sensor network. It consists of a WMA 012 and a monitoring center 300. Medical experts perform analysis of the data in the monitoring center 300 to provide timely medical services. The WMA 012 consists of one or a few sensor nodes and a computing module 200. Sensors in the sensor nodes 100 are adhered to the skin or implanted in the body to collect physiological, activity, environmental, and physiological data. Sensor nodes connect to the corresponding computing module 200. The computing module 200 processes and integrates the information and deduces a physiological status, human activity status, environmental status, and physiological status. The computing module 200 then transfers the data and processing results to the monitoring center 300.

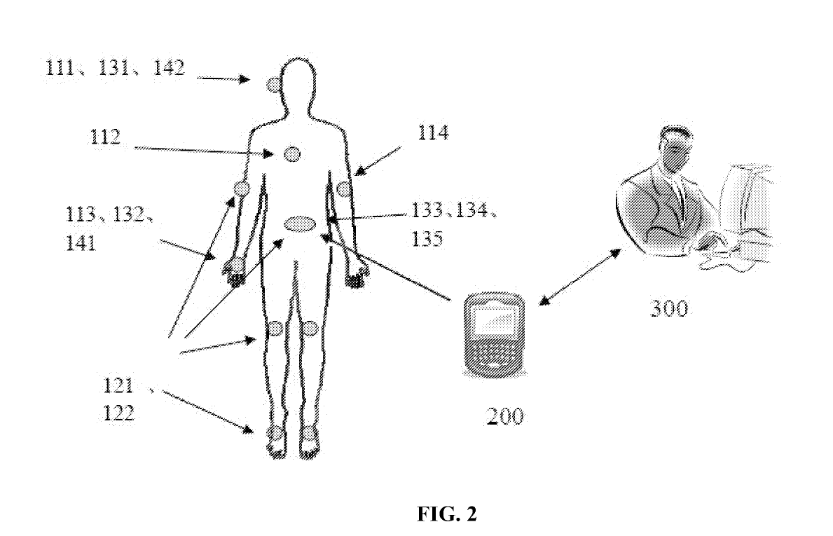

FIG. 2 shows the embodiment of this invention, namely, the RBMS:

Sensor nodes 100 are placed on (or in) different parts of body and consist of:
(1) Physiological sensors: temperature 111, ECG 112, oximeter 113, blood pressure 114, and other sensors, such as EEG, respiration, and blood sugar sensors.
(2) Activity sensors: gyroscope 121, accelerometer 122, etc. Other activity sensors or admeasuring apparatuses are: tensiometers that detect joint motion, video monitoring camera, etc.
(3) Environmental sensors: microphone 131 to detect noise level, light sensor 132, environmental temperature sensor 133, biochemistry 134, GPS 135, etc.
(4) Physiological sensors: skin conductivity sensor 141, microphone 142 to detect events that may affect mood, such as shouting, etc.

The computing module 200 can be implemented on a dedicated device or on a general purpose PDA or smart phone. Sensor nodes 100 collect and preprocess physiological, activity, environment, and physiological signals. After preprocessing, signals are further processed, integrated, classified, and stored in the computing module, and are finally transferred to the monitoring center 300. If an abnormality occurs, either the computing module or the monitoring center will promptly inform a medical center or family member, depending on the alert definition parameters.

The embodiment of this invention will be given in detail:

(1) Sensor Nodes 100

FIG. 2 shows two categories of sensors in the sensor nodes of the WMA: One category includes physiological sensors and the other category includes situational and contextual (environmental) sensors. Situational and contextual factors affect a body's physiological status, and this category of sensor is further divided into three subcategories: activity sensors, environmental sensors, and physiological sensors.

1) Physiological Sensors:
As an important measurement of the body status, physiological signals must be acquired in real-time with good accuracy to provide necessary data for diagnosis, therapy, and health planning. Physiological sensors 110 are applied to the body to collect physiological signals, such as ECG, EEG, blood sugar, blood pressure, and body temperature. The physiological sensors 110 may be worn, adhered to the skin, or implanted in the body. With the rapid development of sensor technologies, new types of sensors and new miniaturized smart sensors will become available.

2) Activity Sensors:
Activity sensors measure motions of the body and are referred to as motion sensors. Motions are one of the key factors affecting human physiological status. Activity type, intensity, and duration are factors related to energy consumption, heart rate, and cardiovascular fitness. The commonly used motion sensors, such as gyroscopes and accelerometers, are placed on the trunk (torso), thigh, and crus. Motions of these body segments may be used to estimate the type, intensity, and duration of an activity. Body segment motion and human activities can also be derived from other sensory measurements, such as video camera data.

3) Environmental Sensors:
Environmental conditions are other key factors affecting physiological status. Environmental factors include: temperature, noise, air conditions, location, etc. High temperatures, loud noises, and severe air pollution can cause changes in the physiological status, and location information may facilitate interpretation of the physiological indicators. Outdoor localization can be realized using GPS, triangulation via multiple wireless communication base stations (For details, please refer to “analysis of WCDMA locating” by Zhang Chong), or via ultrasonic wave or microwave-based radar.

4) Physiological Sensors:
The measurement of physiological status may be achieved by measuring skin conductivity (see M. Strauss, C. Reynolds, S. Hughes, K. Park, G. McDarby, and R. W. Picard (2005), “The Hand Wave Bluetooth Skin Conductance Sensor,” The 1st International Conference on Affective Computing and Intelligent Interaction, Oct. 22-24, 2005, Beijing, China). Events that trigger a bad mood can be recorded using a microphone.

5) Other Factors and Corresponding Sensors.

(2) Acquisition, Processing of Signals and Medical Services

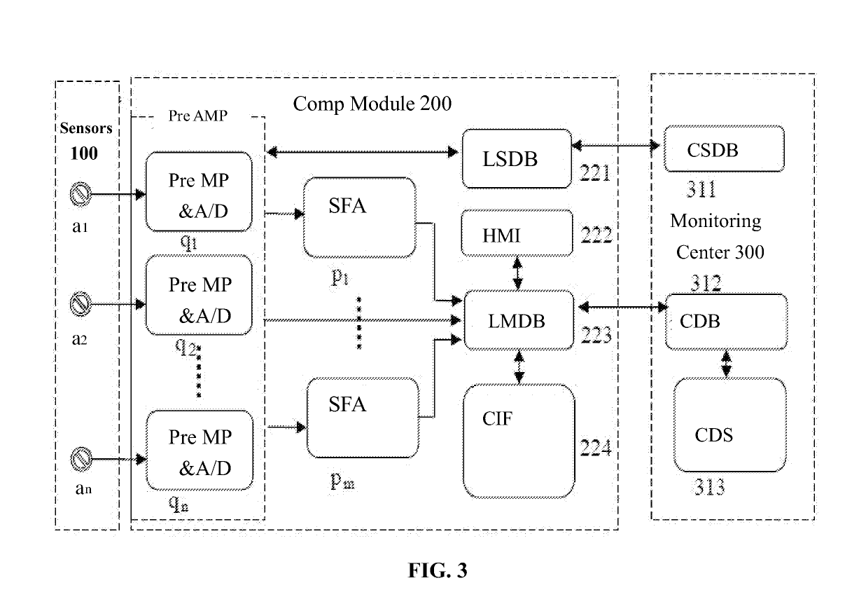

FIG. 3 shows a process flow and block diagram that illustrates the acquisition, processing, analysis, fusion, storage of multiple signals, deduction of bodily health status, and provision of medical services using the RBMS described in this invention. Assume that the sensor nodes 100 of the system consist of a set of n sensors, a₁, a₂, . . . , aₙ, that collect analog signals, some of which are weak. In this case, a set of n preamplifiers and ADCs q₁, q₂ . . . , qₙ, is needed. The analog signal must be amplified to meet the required ADC signal levels. In addition, a reasonable signal-to-noise ratio must be maintained for weak signals, such as the EEG and ECG.

In many cases, it is necessary to place several sensors of the same type on different parts of the body for accurate collection of physiological, activity, environmental, and physiological signals. For example, an ECG apparatus commonly used in hospitals includes 12 electrodes that are adhered to 12 specific locations on the body. For the sake of portability, two or three electrodes may also suffice. Similarly, the monitoring of activity levels requires integrating signals from one of several configurations: for example, a group of three motion sensors positioned on the trunk and thighs; a group of five motion sensors positioned on the trunk, thighs, and crura; or a group of seven motion sensors positioned on the trunk, thighs, crura, and arms. Signals from sensors of the same type that are located on different body segments are integrated to construct a model for the movement and deduce the activity type, intensity, and duration. Thus, SFA p₁, p₂, . . . , pₘ integrate (fuse) signals from sensors of the same type that are placed on different segments of the body to derive a definite status and parameters of the underlined targets. The principle used for signal processing and fusion in SFAs is the principle of signal acquisition and analysis. For example, processing algorithms for ECG signals can detect the heart rate and abnormal waveforms, such as premature beats. This principle is described, for example, in Gari D. Clifford, Francisco Azuaje, Patrick McSharry, Advanced Methods and Tools for ECG Data Analysis, Artech House Publishers, 2006.

Similarly, integration of acceleration data from the accelerometers placed on the trunk and thighs permits estimation of speed and gait parameters, activity type, and intensity, and abnormalities in gait may be identified. For details, please refer to DONG Liang, WU Jian-Kang, BAO Xiao-Ming, Tracking of Thigh Flexion Angle during Gait Cycles in an Ambulatory Activity Monitoring Sensor Network, Vol. 32, No. 6 ACTA AUTOMATICA SINICA November, 2006, pp. 938-946.

The aforementioned integration of signals of the same type from different parts of the body is called “collaborative fusion”: several sensors work together to deduce the activity type, or several electrodes work together to deduce the heart status. This fusion is at the signal level rather than the information level, and therefore referred to as “low-level fusion.”

The monitoring database 223 in the computing module 200, the LMDB, stores raw signals from the ADC and analysis results from the SFA and the CIF. After analysis, only the analysis results corresponding sample signals are stored. It is unnecessary to store all raw signals. For example, if a user sits for 30 min, only the following information is necessary: activity: sit; from s/m/h d/m/y to: s/m/h d/m/y; which corresponds to 128 raw data samples.

To make decisions regarding physiological status, the CIF 224 fuses sensor information from the SFAs and the LMDB 223. The term “information” rather than “signal” or “data” is used here because the input data for 224 are the analysis results from the SFAs. For example, the data may consist of heart rate, abnormalities, activity type, and intensity. Fusion processes in the CIF 224 are fusions (integrations) at the information level, which is higher than the signal level.

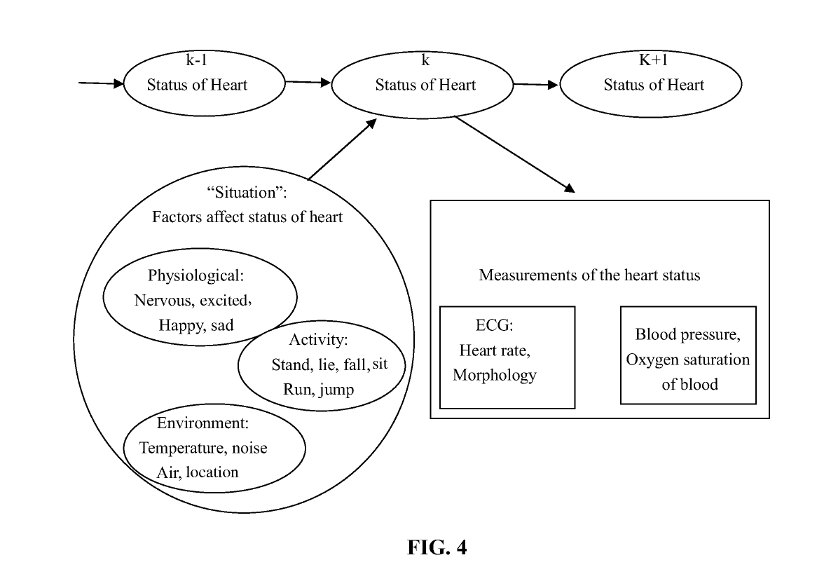

FIG. 4 illustrates the real-time estimation of the heart status using a context-aware fusion method based on Bayesian network dynamic systems theory, which is performed in the CIF. The heart status varies over time. Heart status at time k is related to its previous status at time k−1, i.e., the heart status at subsequent times k+¹ may be predicted according to the current status. On the other hand, various factors may affect the heart status. Here, we consider three factors, namely, activity (sit, lie, stand, walk, run, jump), environment (temperature, noise, air condition, location), and physiological conditions (nervousness, excitation, anxiety, happiness, calmness). All such factors are situational or contextual. In other words, the heart status must be discussed in the situational context. A set of measurements are used to determine the heart status. These measurements include ECG, blood pressure, oxygen saturation in the blood, etc. The heart status, its time evolution, measurements, and situational context constitute a Bayesian network subject to time-dependent dynamics. The links and directionality of links represent the relationships among these variables, the status, measurements, and influencing factors. Defining the Bayesian network using inputs and monitoring the network evolution over time permits estimation of the heart status as a function of time. People are familiar with heart diagnosis protocols using ECG instruments in hospital: a doctor performs an ECG test on a patient while the patient is lying down, then diagnoses the patient according to the results. Under this fixed situation, i.e., lying down on a hospital bed, the effects of situation or context can be ignored. However, in most cases, patients with heart problems cannot be diagnosed in fixed hospital situations because cardiovascular problems occur in daily life and are caused by a variety of situations, such as bad mood, high temperatures, and intense physical activity. Thus, continuous monitoring and context-aware data fusion are key approaches to the issue of heart status diagnosis and will lead to a new generation of medical instruments and novel measurement and diagnosis methodologies.

Estimating the health status of the heart requires long-term observation of a heart's dynamics and the related situational context. For example, ECG and activity measurements may indicate that one's heart rate is 62 beats/min during sleep, 75 beats/min during walking at a speed of 5 km/h, and 100 during running at a speed of 10 km/h. It is standard to conclude that this heart is healthy. A heart rate that increases by either much larger or much smaller percentages during exercise would indicate a poor or worrisome health status. The WMA described in this invention permits measurement and analysis of heart dynamics in terms of both heart rate and ECG waveform changes as a function of activity intensity. This discussion demonstrates that diagnosis is difficult without situational information.

As shown in FIG. 3, two databases are included in the computing module. The LMDB 223 stores sensory data and analysis results, definitions of the alert and reminder parameters, and the thresholds for alerts and reminders. The LSDB stores system information and parameters, such as sensor type, location, sampling rate, and battery level. The duration of storage depends on the storage capacity and ranges from weeks to months.

The large database in the monitoring center server 300, namely, CDB 312 and CSDB 311, stores all user data, including the personal profiles, all data for the full duration of WMA use of a user (analysis results together with samples of the raw signals, heart status, cardiovascular fitness indices, and other diagnostic results over time, statistics over time, including daily and monthly cycles, and types of information, such as the quantity and intensity of exercise during a day, changes in the fitness indices, etc.). The CSDB 311 in the monitoring center 300 stores the system parameters for all WMAs and for the monitoring center system.

The CDB 312 in the monitoring center 300 stores a users' medical history, diagnosis, treatment plan, response to treatment, specific measurements, and the set of service definitions and threshold values.

Synchronization between the two databases at the monitoring center 300 (namely, CDB 312 and CSDB 311) and those in the WMAs 012 (namely, LMDB 223 and LSDB 221) is event-driven. Communication events from 012 to 300 include transfer of new data and analysis results, alerts, reminder triggers, and variations in the system parameters. Communication events from 300 to 012 include updates to a new or existing user's profile, updating the definitions of service, alerts, reminders, alert and reminder threshold values, software upgrades for 012, etc. As a result of synchronization, related reactions may be implemented. For example, when the monitoring center receives an alert, a corresponding reaction would be carried out immediately. Depending on the alert type and other parameter definitions, an alert may be sent to caregivers, family members, or emergency medical services if necessary. In case of an emergency, the information in the patient profile, medical records, and monitoring data may be routed to the emergency center and ambulance. WMAs include instructions from the CSDB for modifying the system configurations of 012 that would be performed immediately when instructions were received via database synchronization.

CDS 313 in the monitoring center 300 is based on the data and information stored in the CDB 312 and on the analysis tools provided by the monitoring center 300. CDB 312 stores the “full information” of each user. By “full information,” we mean long-term physiological (heart, body, brain) status and changes, physiological circadian rhythm and its changes, and the corresponding situational information. The CDS 313 includes two types of function: One function is medical diagnosis and therapy research enabled by the large and comprehensive database that gathers information on a variety of users. Although information fusion is conducted in individual CIFs, novel methods for context-aware dynamic measurements, medical interpretations, diagnosis, and treatment may be researched and validated by a variety of medical practices. For example, the chairman of the American Heart Association (AHA), Prof. Philip F. Binkley showed in clinical research that the variations in heart rate that occur over 24 hour periods, as a function of activity levels, serve as good indicators of some diseases, such as myocardial infarction, myocardial atrophy, and deadly arrhythmia. The 24 hour heart rate pattern variation as a result of activities can be used to select the best time of day for administering medicine or treatments. The 24 hour blood pressure pattern variations can be used to predict diseases, such as deadly high blood pressure. The other function of the CDS is to build a profile for each user and to provide prompt personalized service.

The HMI 222 in a WMA 012 has following functions: a clock function for including data timestamps or stopwatch functions; signal process and analysis functions that enable the real-time display and retrieval of current or past data and analysis results and to provide advice; networking functions for access to the online community and for uploading, modifying, deleting data, and for interacting with caregivers, professionals, or friends; system functions for maintaining, upgrading, and self-organizing. The WMA 012 allows plug-and-play capabilities for sensors. The system will be able to automatically adjust system operation to optimize performance in the event of a change in sensors or other system components.

The function that enables self-tuning in response to system configuration changes is implemented by the LSDB and by data analysis programs and applications in the WMA 012 management system. Adding or deleting sensors will be noted in the LSDB 221, database entry changes trigger loading of the appropriate data analysis programs into the system and trigger parameter updates. For example, the analysis programs for data from one, three, or five accelerometers in a WMA differ greatly. The analysis algorithms that deduce activity type and gait parameters are very different for data streams that include one, three, or five accelerometers. Therefore, the analysis programs and applications must be chosen according to the number of accelerometers.

Additionally, the selection and modification of application programs may vary from person to person. For example, when used for exercise monitoring, the application program of the WMA 012 would read a user's profile and suggest an optimal exercise regimen based on the individual's lowest and highest heart rates. The optimal duration time is defined and a reminder is sent to the individual when the predefined limits are met.

(3) System Structure

In terms of hardware implementation, sensor nodes 100 and computing module 200 in FIG. 3 in the WMA 012 shown in FIG. 1 may be implemented in a variety of ways. As a result, RBMS includes several system structures.

Sensors in sensor nodes 100 in a RBMS, especially physiological sensors, may be implanted inside body, whereas most sensors are adhered to, bound to, or embedded in accessories such as clothes, hats, shoes, gloves, corsets, watches, earphones, or are adhered to body in other manners.

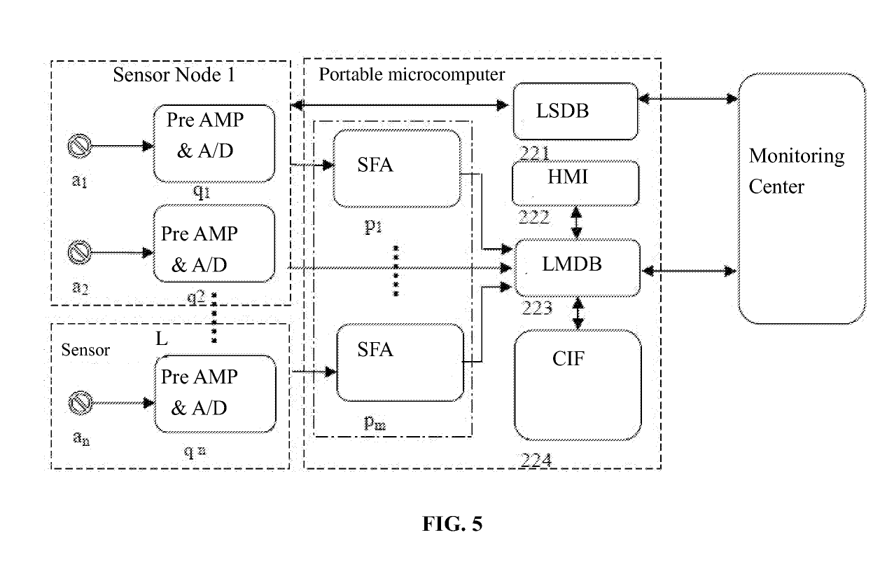

FIG. 5 illustrates an implementation scheme for the RBMS:

This scheme consists of one or a few sensors. Sensors, preamplifiers and ADCs, wired/wireless communication, controller, and power management together form a sensor node in the form of an embedded system module, or even as a single system on a chip. An independent sensor node is capable of collecting, temporarily storing, and transmitting signals via either wired or wireless communication protocols. If the sensor node is sufficiently powerful, the SFA may be integrated in the sensor node to reduce the communication load, because only the processing results (as opposed to the full raw dataset) need be transmitted. Inside of the PDA or smart phone are implemented the LSDB 221, LMDB 223, and CIF 224 of the computing module.

PDA or smart phones communicate with sensor nodes wirelessly, via, for example, Bluetooth or Zigbee. In this case, the complete WMA is a “wireless body sensor network,” and the PDA or smart phone serves as a gateway. Each sensor node performs time synchronization with the gateway, communicating using the TDMA protocol.

Another implementation scheme of RBMS includes all sensors connected directly to wearable microcomputers that may be specially designed. As a result, the complete WMA is an embedded system.

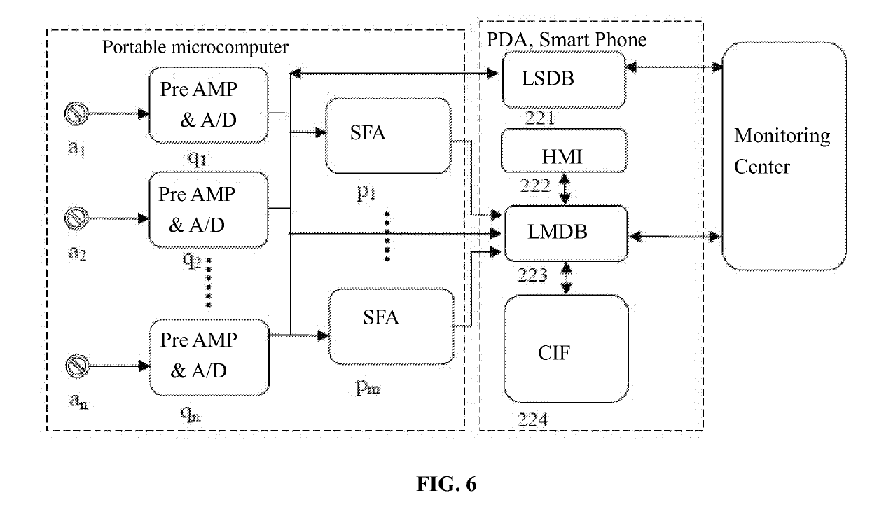

Yet another implementation scheme of the RBMS is shown in FIG. 6, in which a single powerful sensor node includes implementation of most of the units of the computing module 200. A PDA or smart phone is used to implement the user interface HMI, local databases, and communication modules.

The complexity of an RBMS depends on the type and number of sensors used. If only one aspect of the body status is monitored, the system may become:
1) an ECG or blood pressure continuous monitoring and analysis apparatus, which is portable and capable of transferring data to the monitoring center. This system is different from the Holter in that it connects users and caregivers.
2) Activity monitoring apparatus through a set (1, 3, 5, or more) of accelerometers. Types, intensity, and duration of activities may be derived from the accelerometer data. On the one hand, the type, intensity, and duration of activity can be used to estimate energy consumption, to provide advice for exercise and weight loss. On other hand, activity data may be used to monitor the quantity of daily activity according to health-based daily activity rules, and the long-term variations in activity can be derived. Such information is important for health care, especially in research and application of elderly care. For example, a decrease in activity, a change in the time at which a person gets out of bed, unusually long periods of walking, etc. all suggest potential problems.
3) Environmental monitoring apparatus. The portability and location tracking capabilities have many applications. For example, a localization apparatus may be worn by children to assist parental supervision.
4) Physiological admeasuring apparatus using skin conductivity and EEG signals could improve sleep quality or trace the physiological status of soldiers at the front lines.
The WMA may be operated independently without a monitoring center. The functions of data acquisition, processing, fusion, human-machine interaction, wired/wireless communication, processing results may be used to send reminder and alert messages to users, family members, or caregivers. The WMA can incorporate self-diagnosis and self-organization capabilities.
Different combinations of sensors apply to different applications. A simple application is given as follows.

A Simple Application
RBMS could usher in a new generation of medical equipment that expands healthcare and medical services from hospitals and clinics to the community and family. Monitoring and care advice is available during daily activities, at work, and during leisure time. As a simple example, consider a simple WMA consisting of an ECG and three accelerometers on the waist and legs. The sensors are packaged into one or two wireless sensor nodes. The nodes receive ECG and acceleration signals. After amplification and ADC, the nodes send signals to a smart phone on which the computing module is implemented.
The smart phone processes the ECG and accelerometer signals. The results of the ECG analysis are heart rate and abnormal beat detection, such as premature beats. Analysis of the three accelerometer signals indicates the type of activity: 1) static (standing, sitting, lying down); 2) walking and step frequency (walk, run, upstairs, downstairs); 3) transition (standing up, sitting down, getting up). All results and related raw data are stored in the LMDB inside the smart phone.
For example, suppose a 60-year-old WMA user is jogging. The smart phone detects and records the fact that the user's speed is 6 km/hour for 10 minutes. The smart phone monitors the user's heart rate variations for abnormal events and simultaneously computes the energy consumption. When the user speeds up to 8 km/h, his heart rate reaches a saved threshold and the smart phone alerts the user to slow down. 25 minutes later, the user's energy consumption targets are met. The smart phone indicates this fact to the user and suggests that exercise be ceased.

Meanwhile, two premature beats were detected and recorded in the ECG waveforms and activity intensity information. This is not yet an arrhythmia, but the smart phone sends the information to the user's doctor as a precaution. The doctor receives this record of recent heart rate variation in the context of the activity data. This data is also part of a larger dataset that includes daily activity statistical data, the user's daily schedule, and variations in all parameters. The doctor can then make an informed assessment of the premature beats, prepare advice for the patient, and send this advice to him.

(4) Online Health Community

Variations in the uses of the WMA 012 may be made by reducing the type and number of sensors, simplifying the processing function, and focusing on its wearability for considerations of style. A variety of applications may be envisioned, for example, exercise management, weight loss, and elderly care. We refer to this type of WMA as a new lifestyle apparatus (NLA).

As an example, consider an NLA consisting of an ECG 112, one accelerometer 122, and a respiration sensor 133. Clock and stopwatch functions are embedded in the system. From the ECG measurements, abnormal signals, such as premature beats, can be detected if present. From the accelerometer 122, activities may be classified, the intensity and duration may be calculated, and the energy consumption may be estimated. The effect of exercise and weight-loss efforts on body health status can be evaluated through fusion and long-term continuous analysis of ECG, respiration, and activity data and their variations, especially heart and respiration dynamics during exercise. Refer to the AHA exercise standard for testing and training for details. Use of an NLA allows a person to evaluate his or her own cardiovascular fitness, and plan an exercise regimen, daily schedule, and weight-loss scheme accordingly.

Fitness assessments encompass a variety of measures that are designed to provide individualized feedback regarding one's overall fitness status and/or physiological responses to physical effort. Cardiovascular fitness is an important indicator. The most commonly used cardiovascular fitness tests can be conducted simply using an NLA. For example, in the Rockport one-mile running test, testers try their best to finish a one-mile run, and the time and average heart rate is recorded. According to the testers' gender, age, weight, the cardiovascular fitness index, the VO₂ₘₐₓ can be calculated as follows:

VO₂ₘₐₓ(ml·kg⁻¹·min⁻¹)=88.768+8.892×(Gender,male=1,female=0)−0.21098×(Weight,kg)−1.4537×(Time,min)−0.1194×(Heart Rate,per Min)

Similarly, other health fitness assessments are available through this approach. With the help of health fitness monitoring, the health status of users, or of a group of users, such as students, can be evaluated. Exercise and weight-loss regimens could be devised according to individual needs.

Exercise and slimming guidelines can be devised very simply using an NLA. For example, the lowest and highest heart rate can be calculated by NLA according to a user's profile and exercise record:

Lowest HR=(220−age−Rest HR)×50%+Rest HR

Highest HR=(220−age−Rest HR)×70%+Rest HR

Where the selection of 50% or 70% depends on personal health conditions. During exercise, the NLA will notify the user when the heart rate limits are reached.

An online health care and fitness community is constructed by the RBMS on the monitoring center server. Community members create accounts and storage space for is allocated to a user as soon as an NLA is activated. The community server provides a set of data analysis tools and software updating services to NLAs. NLAs automatically upload data using database synchronization functions. The user can log on to the community, view and analyze his own data, view health news and new findings in the community, share data and ideas with other members, search for friends who may suffer from similar problems, and talk with an expert.

While particular embodiments of the invention have been shown and described, it will be obvious to those skilled in the art that changes and modifications may be made without departing from the invention in its broader aspects, and, therefore, the aim of the appended claims is to cover all such changes and modifications as fall within the true spirit and scope of the invention.

The invention claimed is:

1. A real-time body status monitoring system (RMBS), comprising:
a sensor node, which is attached to the body of a user and falls into one of two categories of sensors: physiological sensors or contextual and situational sensors;
a computing module connected to the sensor nodes, which provides a means for acquiring multiple sensor signals, performing multiple-sensor signal fusion and analysis for sensors of the same type placed on different locations of the body, performing context-aware fusion of physiological and contextual information, storing analysis results and sample data in a database, interacting with the user, and communicating with the monitoring center; and
a monitoring center that connects to and serves many WMAs wirelessly or via a network infrastructure, forming a complete RBMS, a means for receiving and storing data in the central database, providing a platform for further data analysis across users, time, and data modalities, providing a platform for consultation services and real-time emergency response, and for prompt connections between users, caregivers, and family members;
wherein,
one computing module and one or a few sensor nodes constitute a WMA worn by a user.

2. The real-time body status monitoring system (RMBS) of claim 1, wherein
the physiological sensors that are attached to the user's body include, but are not necessarily limit to, heart rate meters, electrocardiograms (ECGs), sphygmomanometers, blood oxygen saturation meters, thermometers, respirometers, electroencephalographs, and blood glucose meters;
said contextual and situational sensors consist of three subcategories of sensors:
(1) activity sensors, including, but not necessarily limit to, accelerometers, microgyroscopes, tensiometers, and video cameras;
(2) environmental sensors, including, but not necessarily limit to, temperature sensors, acoustic sensors that measure the noise level, and location sensors; and
(3) physiological sensors, including, but not necessarily limit to, skin conductivity sensors, electroencephalogram sensors, and microphones.

3. The real-time body status monitoring system (RMBS) of claim 2, wherein said sensor attachment means include, but are not necessarily limited to, pasting, binding, and embedding in accessories, such as clothes, hats, shoes, gloves, corsets, watches and earphones.

4. The real-time body status monitoring system (RMBS) of claim 1, wherein said computing module consists of:
a) a set of preamplifiers and analog-to-digital converters, a means for receiving signals collected by sensors, amplifying those signals, then converting the signals to digital signals;
b) a set of sensor signal fusion and analysis units (SFAs), which fuse and analyze signals from the same type of sensor placed on different parts of the body; the units then send the processed results to the local database; the processed results can be used as input for context-aware multi-sensor information fusion units (CIFs), or used by the user, caregiver, or a family member directly;
c) a CIF, which provides a means for receiving results from the SFAs and fusing physiological information in the context of activity, environment, and physiological status to estimate the health status of the user by means of Bayesian network dynamic theory;
d) a human-machine interaction unit (HMI), which provides a means for displaying the results from SFAs and CIFs, receiving and responding to the requests of users, and displaying information from the monitoring center;
e) a local monitoring database unit (LMDB), which provides a means for storing data over the course of weeks and months; here, the data includes sensor raw data, results from SFAs and CIFs, personal profile data, medical history information, and parameters and thresholds that define alerts and reminder criteria; and
f) a local systematic database unit (LSDB), which provides a means for storing configurations and runtime parameters for the WMA.

5. The real-time body status monitoring system (RMBS) of claim 1, wherein
said monitoring center consists of:
a) a context-aware diagnosis and service unit, which provides a platform that supports sets of analysis tools for fusing and mining information across users, time, and data modalities to find rules and regularities for context-aware diagnosis and therapy in daily life, and providing a platform for prompt medical and consultation services;
b) a central database, means for storing all gathered information, including results from the WMA together with samples of raw sensor data, a user's personal profile information, medical records, the diagnosis results, treatment plan, and therapy results; and
c) a central system management database (CSDB) and software administrative means for storing all system parameters for the complete RBMS.

6. The real-time body status monitoring system (RMBS) of claim 4, wherein once the threshold values are reached, alerts or reminders will be triggered according to predefined rules in the LMDB.

7. The real-time body status monitoring system (RMBS) of claim 4, wherein the LSDB receives commands from the monitoring center and modifies the system parameters of WMAs appropriately.

8. The real-time body status monitoring system (RMBS) of claim 4, wherein the central database, CSDB, and software administration synchronize with the LMDBs and LSDBs bidirectionally in an event-driven manner LMDBs initiate synchronization with the central database if new data and analysis results are present; LSDBs initiate synchronization with the CSDB and software administration if hardware or system changes are required for the WMA; the central database initiates synchronization with LMDBs if the alert or reminder parameter sets or medical instructions change; the CSDB and software administration initiates synchronization with the LSDBs when any system commands are issued.

9. The real-time body status monitoring system (RMBS) of claim 4, wherein said WMA consists of a computing module and one or several sensor nodes, and these units are connected via either wired or wireless communication, and the computing module communicates with the monitoring center;
a) the sensor node is an embedded system or a system on a chip that consists of one or several sensors, preamplifiers, and analog-to-digital converters, wired/wireless communication, microcontrollers, and power management;
b) a computing module is implemented on a dedicated microcomputer or off-the-shelf personal digital assistant (PDA) or smart phone to include the SFAs, CIF, LMDB, LSDB, and HMI; and
c) SFAs can be implemented in a sensor node or computing module; depending on the computing capabilities of the sensor node, the quantity of data transmitted may be greatly reduced if analysis functions are implemented within the sensor node.

10. The real-time body status monitoring system (RMBS) of claim 4, wherein the second implementation includes sensors that are directly connected to the computing module, which is implemented in a portable microcomputer, PDA, or smart phone, and in which all data processing is implemented; the computing module is also connected to the monitoring center wirelessly or through a network infrastructure.

11. The real-time body status monitoring system (RMBS) of claim 4, wherein yet another implementation option includes extension of the computing power of sensor nodes to include all data processing units, including SFAs and CIF; this “powerful sensor node” connects to a PDA or smart phone, which acts as the user interface, data storage, and communication intermediate with the monitoring center.

12. The real-time body status monitoring system (RMBS) of claim 4, wherein the SFAs achieve significant data interpretation via processing, analyzing, and fusing signals from sensors of the same type that are located at different parts of the body; for example, identifying activity type, intensity, and duration by fusing acceleration signals from different part of the body, calculating heart rates and detecting abnormal waveforms in the ECG signals from various electrodes.

13. The real-time body status monitoring system (RMBS) of claim 4, wherein contextual/situational factors affect the body physiological status, including activity, environment, and physiological factors; wherein context-aware information fusion means the evaluation of the body status according to physiological measurement in the context of situational information.

14. The real-time body status monitoring system (RMBS) of claim 5, wherein context-aware service means services based on the analysis and results of long-term recording of physiological status, physiological circadian indicators, variations of these indicators, respective contextual information, derivations from an individual's data and across users and age groups.

15. The real-time body status monitoring system (RMBS) of claim 1, wherein when the monitoring center is unavailable, a user can acquire body status information and receive alerts and reminders from the WMA, transfer crucial data to caregivers and family members; meanwhile, all data, including the raw data and processed results, are stored for weeks or months within the WMA.

16. The real-time body status monitoring system (RMBS) of claim 1, wherein the WMA becomes a specific apparatus when only one type of sensor is included:
a) the WMA becomes a dynamic heart monitor if only an electrocardiograph sensor is included;
b) the WMA becomes an activity monitor if only accelerometers and gyroscopes are included; the monitor can be used for continuous activity monitoring, activity identification, quantitative analysis, energy consumption calculations, and exercise planning;
c) the WMA becomes a localizer, if only a localization sensor is included; and
d) the WMA becomes a mood meter, if only skin conduction sensors are included.

17. The real-time body status monitoring system (RMBS) of claim 4, wherein human-machine interactions include functions such as timing, display of information processing and analysis results, network-based interactions, system maintenance and update, and self-organization; it can be used to select, set, modify, and run application programs according to variations in the sensor configuration.

18. The real-time body status monitoring system (RMBS) of claim 1, wherein the system can be simplified to a health monitoring and consultation apparatus in which part or all sensors in the group, ECG sensors, accelerometers, respiration sensors, and environmental thermographs, are used; through use of the apparatus, a cardiovascular health index test can be performed to devise, guide, and monitor an exercise plan; the heart status can be monitored during exercise for safety.

19. The real-time body status monitoring system (RMBS) of claim 18, wherein, the apparatus can access the online community, which forms a platform for communication and consultation; in the community, user accounts are created, storage space is allocated, and data analysis tools are provided; users can communicate directly with professional clinicians online or leave messages; they can communicate with other uses and form interest groups.

20. The real-time body status monitoring system (RMBS) of claim 1, wherein, the community is accessed by the WMA wirelessly; the WMA can upload data to the user's account automatically, download and upgrade new software and analysis tools, receive medical advice and messages; the user can manage his own account and analyze his own data using the tools provided.
